(19) United States
(12) Patent Application Publication
(10) Pub. No.: US 2022/0270761 A1
(43) Pub. Date: Aug. 25, 2022

(54) Systems and methods for automated generation of personalized health screening recommendations

(71) Applicant: Simon Bloch, Mountain View, CA (US)

(72) Inventor: Simon Bloch, Mountain View, CA (US)

(21) Appl. No.: 17/566,552

(22) Filed: Dec. 30, 2021

Related U.S. Application Data
(60) Provisional application No. 63/153,334, filed on Feb. 24, 2021.

(51) Int. Cl. G16H 50/30; G16H 10/20; G16H 10/60; G16H 50/20

(52) U.S. Cl. CPC G16H 50/30; G16H 10/20; G16H 10/60; G16H 50/20

(57) ABSTRACT

An automated personalized health screening recommendation system based on a curated guideline-based database and an interactive subject input is configured to match personal information to a plurality of health screening guidelines. A software-based recommendation engine may be used to process recommendations to produce one or more cumulative health screening recommendations as a health screening report. Further embodiments describe follow-up and tracking of the subject compliance with recommended screenings and adjustments to the screening types and schedule as the personal information changes.

### CROSS REFERENCE DATA

This patent application claims a priority date benefit from a U.S. Provisional Patent Application No. 63/153,334 filed 24 Feb. 2021 entitled CLINICAL DECISION SUPPORT SYSTEM AND METHOD FOR AUTOMATED AND PERSONALIZED HEALTH SCREENING RECOMMENDATIONS, which is incorporated herein in its entirety by reference.

### BACKGROUND

Without limiting the scope of the invention, its background is described in connection with automated aids to clinical decision-making. More particularly, the invention describes methods and systems for automated determination of appropriate types and schedules for health screenings based on computer-implemented recommendation software.

People in modern life are exposed daily to a variety of health risks resulting from a worsening environment, stress, lifestyle choices, and food consumption. This exposure contributes to silent disease risks. Silent diseases are diseases that do not exhibit obvious symptoms or signs but can progress to advanced stages before they are discovered.

The result of silent diseases is an increased rate of risk for a variety of ailments such as
heart and cardiovascular diseases including high blood pressure, abnormal cholesterol, coronary artery disease, aneurysms, and stroke;
various cancers such as breast, cervical, prostate, colon, skin and other cancers;
various metabolic diseases, such as diabetes, high glucose, liver, kidneys;
digestive disorders, such as acid reflux, celiac disease, etc.;
general health anomalies and conditions, such as immune system disorders and inflammation;
mental health issues, such as depression, anxiety, various addictions;
long-term infections, such as hepatitis, HIV, sexually transmitted disease, and many others.

Regular health screenings can identify exposure to these and other types of silent diseases. Screening tests are designed to detect diseases or risk factors for a disease before symptoms appear. Many of the silent diseases are preventable and curable if identified and intervened in time. Identifying risk factors for the disease may reduce the chance of developing certain diseases or prevent them completely. Early detection is crucial to treating cancers, preventing heart attacks, and controlling many other chronic issues. Diseases can be identified and treated through regular health screenings, which are important for people of all ages.

People often ignore caring about their health until there are visible symptoms, at which point, care is more complex. Many people understand the importance and benefits of health screenings, yet many still choose not to undergo a health screening. For example, only eight percent of US adults ages thirty-five and older had received all the high-priority, appropriate clinical preventive services recommended for them. The dominant reason for avoiding screenings is not knowing which screenings to get, as well as when and why these screenings are important. There are hundreds of screenings available, and medical guidelines include several dozen screenings that each person should receive.

Certain screening guidelines can be complex. For example, U.S. Preventive Services Task Force (USPSTF) recommends annual hypertension (high blood pressure) screening for adults aged 40 years or older and for those who are at increased risk for high blood pressure. Persons at increased risk include those who have high-normal blood pressure (130 to 139/85 to 89 mm Hg), those who are overweight or obese, and African Americans. Adults aged 18 to 39 years with normal blood pressure (<130/85 mm Hg) who do not have other risk factors should be rescreened every 3 to 5 years.

In another example, USPSTF recommends lipid profile screening for lipid disorders, also called dyslipidemias, that are abnormal elevations of total cholesterol, LDL, or triglycerides. These disorders can be acquired or familial. This recommendation applies to adults aged 20 and older who have not previously been diagnosed with dyslipidemia. Increased risk, for the purposes of this recommendation, is defined by the presence of any one of the following risk factors: diabetes, previous personal history of coronary heart disease or non-coronary atherosclerosis (e.g., abdominal aortic aneurysm, peripheral artery disease, or carotid artery stenosis), a family history of cardiovascular disease before age 50 in male relatives or age 60 in female relatives, tobacco use, hypertension and obesity (BMI 30).

Both silent diseases presented in the examples above—hypertension and dyslipidemias—are risk factors contributing significantly to the overall onset and incidence of heart diseases, stroke, and death.

Some screening guidelines can be confusing. As an example, in 2009, the United States Preventive Services Task Force changed the recommended age to initiate mammogram screening in women of average risk from age 40 to age 50. Many high-profile organizations such as the American Cancer Society, American Medical Association, and the National Comprehensive Cancer Network continued to recommend screening initiation at age 40, leaving many women between ages 40 to 49 confused about how to process the new recommendation. Adding further to the confusion, in 2016, The American Cancer Society switched its recommendation to age 45 while further recommending that women can start screening at age 40 if they so choose.

Some screening guidelines are also evolving. For example, colorectal cancer is considered to be a major public health problem. It is the third most frequent cancer at a global level and the fourth most frequent cause of death. The most recent guidelines of The American Cancer Society (2018) recommend that adults aged 45 years and older with an average risk of colorectal cancer should undergo regular screening. All the guidelines have considered four screening options as the most preferred ones: fecal occult blood testing (annual or biennial), fecal immunochemical test (annual), flexible sigmoidoscopy (every 5 years), and colonoscopy (every 10 years). However, there are many evolving discussions amongst health care providers with regards to which tests should be preferred for screening, due to the evolving clinical evidence studies in this field.

The existing complexity, confusion, and evolution of the screening guidelines demand a substantial amount of research to be invested by people that are interested in knowing which screenings they should pursue and when. The limited amount of time and education that people possess in understanding such screening guidelines, leaves them without the ability to engage in preventative and regular screenings that can identify silent diseases.

Some health organizations publish general population screening guidelines and charts that are based on the gender and age of people. These guidelines and charts have limitations. For example, these charts often lack the information to address the personalized nature of each individual and can lack information of substantial influence of ethnicity, medical history, lifestyle, and family history.

Additionally, decisions around screening have been made more difficult owing to the proliferation of preventive screening guidelines by advocacy groups, professional organizations, and government agencies, often with conflicting recommendations on the same topic. Physicians and patients have their own clinical and personal experiences related to health conditions that shape their beliefs about the value of preventive health screening for different conditions.

Decision-making around preventive health screening by family physicians, other health care practitioners and patients have become increasingly complex and controversial in recent years.

Preventive health screening has long been advocated as one of the most important health care strategies to facilitate early diagnosis and treatment, improve quality of life, and prevent premature death. For many years, professional organizations, patient advocacy groups, and clinicians used a combination of public policy, persuasive advertising, and direct clinical messages to patients as methods to increase uptake of screening in specific patient populations and with individual patients. People have been consistently told that the more proactive you are about being screened and the more frequently that you are screened, the healthier and more protected you will be. This belief has become deeply entrenched in society and has been reinforced by both empirical and anecdotal evidence.

Health information management is an overwhelming task faced by people and physicians, who must evaluate a great deal of data to plan a course of action and make decisions. In the case of health screenings, the data must be evaluated according to certain health guidelines. While health guidelines are followed during the decision-making process to the extent possible to avoid missing details that aid in making the decision. Subjective judgments are being made regarding the parameters of the situation due to the large amounts of data that must be processed, and the great number of criteria (dictated by the guidelines) involved in many decisions. Further, not all available data is pertinent to the decision, and much time and effort can be involved by not putting aside this irrelevant data early in the decision-making process.

The health care system in the United States has historically been oriented more toward diagnosis and treatment, relying heavily on medications and treatment procedures, although that is recently starting to change. Increasingly, people and health care systems need to know how well we are screening people for preventable conditions. Meanwhile, the guidelines become more refined, as new evidence emerges, and increasingly require both expertise and experience to interpret them in ways that will benefit people the most. Good health screening guidelines, however, do not necessarily translate into useable and practical tools. Guidelines have become increasingly complex, and the amount of data processed has grown to overwhelming proportions. For example, instead of using simple checklists to trigger standard health screenings for all patients of a certain age and gender, health care practitioners must consider a wide range of health risks, the presence of which can mandate changes in the content, timing, and frequency of appropriate medical health screenings.

To be most effective, decision-makers and physicians, need practical strategies for applying guidelines in daily practice. The need, therefore, exists for new tools to facilitate data assessment, documentation of determinations, and individualized implementation of practice guidelines in an objective manner. A system that could create a useful tool, based on given guidelines, to aid in such determinations or implementations would therefore be desirable.

The need also exists for new tools facilitating follow-up and tracking of patient compliance with suggested types and schedules of appropriate health screenings.

### SUMMARY

Accordingly, it is an object of the present invention to overcome these and other drawbacks of the prior art by providing novel methods and automated tools to determine cumulative personalized health screening recommendations for a subject.

It is another object of the present invention to provide novel methods and tools to combine multiple available health screening recommendations and generate a single report with a personalized recommendation for the types of appropriate screenings for the subject along with the appropriate frequency of each screening.

It is a further object of the present invention to provide novel methods and tools for generating reports with suitable health screening recommendations taking into account a variety of personal data provided by the subject.

It is yet a further object of the present invention to provide novel methods and automated tools to encourage and track compliance with health screening recommendations and update such recommendations from time to time in case of guideline updates or a change in the personal data of the subject.

An exemplary method for providing a personalized health screening recommendation for a subject may include at least the following steps of:
a. providing a computerized recommendation engine operatively connected to a curated guideline database. The curated guideline database may be compiled from a variety of reputable sources and configured to allow matching of personal data input to a plurality of health screening guidelines,
b. soliciting personal data of the subject as input entry via an interactive user interface, the personal data may include more than a conventional set of age and gender. In embodiments, the input may include at least one or more personal information categories such as age, gender, ethnicity, personal medical history, family history, lifestyle, and genetics, with at least some personal information categories including a subset of additional personal data,
c. operating the recommendation engine to automatically process personal data of the subject solicited in step (b) against the curated guideline database to determine types, frequencies, and appropriate schedules of individual health screenings personally recommended for the subject, and
d. combining types and schedules of individual health screenings from step (c) to provide a cumulative personalized health screening recommendation for the subject, such as in one example a calendar with marked-up health screenings.

An exemplary computer system for providing a personalized health screening recommendation for a subject may include:
a. at least one or more processors operatively connected together and to a computer memory. The one or more processors, as well as the computer memory, may be combined into a single computing device or be in part or entirely cloud-based. The computer memory may include a computer-readable program code embodied in the computer memory and executable by the at least one processor to perform operations of a computerized recommendation engine,
b. a curated guideline database configured to match personal data input to a plurality of health screening guidelines, the curated guideline database is operatively connected (remotely or directly) to the at least one processor or may also be cloud-based,
   wherein the computer-readable program code may be further configured to perform at least the following operations:
i. record personal data of the subject as input entry via interactive user interface of at least one processor, the personal data may include at least one personal information category such as age, gender, ethnicity, personal medical history, family history, lifestyle, and genetics,
ii. operate the recommendation engine to automatically process personal data of the subject against the curated guideline database to determine types, frequencies, and schedules of individual health screenings personally recommended for the subject, and
iii. combine types and schedules of individual health screenings from determined in the preceding step to provide a cumulative personalized health screening recommendation for the subject.

An exemplary non-transitory computer-readable storage medium of the invention may comprise computer-readable program code embodied in the medium that is executable by at least one processor to perform the following operations:
a. recording personal data of the subject as input entry via for example an interactive user interface, the personal data comprising at least one personal information category such as of age, gender, ethnicity, personal medical history, family history, lifestyle, and genetics,
b. operating a computerized recommendation engine operatively connected to a curated guideline database to match personal data input from the preceding step to a plurality of health screening guidelines so as to determine types and schedules of individual health screenings personally recommended for the subject, and
c. combining types and schedules of individual health screenings from the preceding step to generate a cumulative personalized health screening recommendation for the subject.

A cumulative recommendation report may be displayed or emailed to a subject and may include information related to what screenings are to be performed, a schedule of the screenings that are to be performed, and at what frequency these screenings are to be performed.

According to at least some embodiments, the screening report may include structured screening information by health categories. The health categories may include heart health, cancer, general health, behavioral health, infections, and immunizations.

The interactive subject input and interface software may be used to solicit an online interview of the subject or may include information on a computer display to directly input the information into the automated tool of the invention. The computer-based or online interview of the subject may include a series of questions, including questions about the subjects' gender, age, ethnicity, medical history, lifestyle, family history, and/or a history of prior health screenings.

In response to the input of the subject, the recommendation engine guided by a curated guidelines database may calculate and determine the most appropriate personalized screenings and screening frequency. The recommendation engine may merge recommendations with scientific and medical educational content as well as provide references to guidelines from reputable health organizations.

The method of the invention may further comprise a step of requiring the subject to accept the terms and conditions of an interview.

The method may further include a step of processing at least one Boolean equation by the recommendation engine for at least one screening, such as a heart health screening in one example.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1 illustrates a block diagram of a system architecture of an automated personalized health screening recommendation system;

FIG. 2 illustrates a flow chart of one example of a method of an automated personalized health screening recommendation system;

FIG. 3 illustrates a detailed block diagram of a system architecture of an automated personalized health screening recommendation system;

FIG. 4 illustrates a flow chart of the method for developing screening recommendations in accordance with the present invention, such as the system and method illustrated in FIGS. 1 and 2;

FIG. 5 illustrates a process of subject interview and inputs in accordance with the present invention;

FIG. 6 illustrates cumulative screening recommendations produced according to the invention;

FIG. 7 is an exemplary illustration of a specific screening presentation; and

FIG. 8 an exemplary illustration of a screening report card of the invention.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

The computerized systems and methods of the invention automate and personalize guideline-based health screening recommendations, including what health screenings a person needs, when to get health screenings, how often one should get health screenings, and provide additional content related to each recommended health screening. The recommendations are tailored to a particular subject situation through an interactive user interface—either via an online program or as a direct computer input. In one preferred arrangement, the system and/or method matches relevant subject data with data extracted and curated from a comprehensive set of clinical guidelines for health screenings and formulates a recommendation to aid decision-making on a course of appropriate action.

Specifically, the computerized system may generate automated and personalized health screening recommendations and a screening report. Health screening recommendations include a list of screenings with information as to what screenings are needed to be performed and when these screenings should be initiated. In one arrangement, the screening report provides supplementary curated science-based educational content about the nature of screening and references to guidelines from top scientific US health organizations. The screening report can also serve as a checklist for a subject's self-care. Alternatively, or in addition, the screening report can be used in a consultation discussion in the next visit with a physician.

The cumulative health screening recommendation report may be structured by health categories, such as heart health, cancer, general health, behavioral health, infections, and immunizations. In one arrangement, a total of three dozen most important health screening guidelines may be considered for the purposes of formulating a cumulative health screening recommendation for a subject and more health screening guidelines may be added over time. Each health screening category may contain information about this screening as well as additional or related health screenings, such as a description of the screening, details about the screening, how the screening is performed, who should be screened and how frequently, what is the screening type, and references to additional guideline information from reputable health science organizations and sources.

In one arrangement, screening recommendations may be made based on input provided by the subject over an online interactive interview. Subject input may include personal data including predefined personal information categories such as gender, age, ethnicity, medical history, lifestyle, and family history. Additional data may be provided within at least some of the personal information categories, such as the medical history may include information about a history of previous vaccinations or previous health screenings.

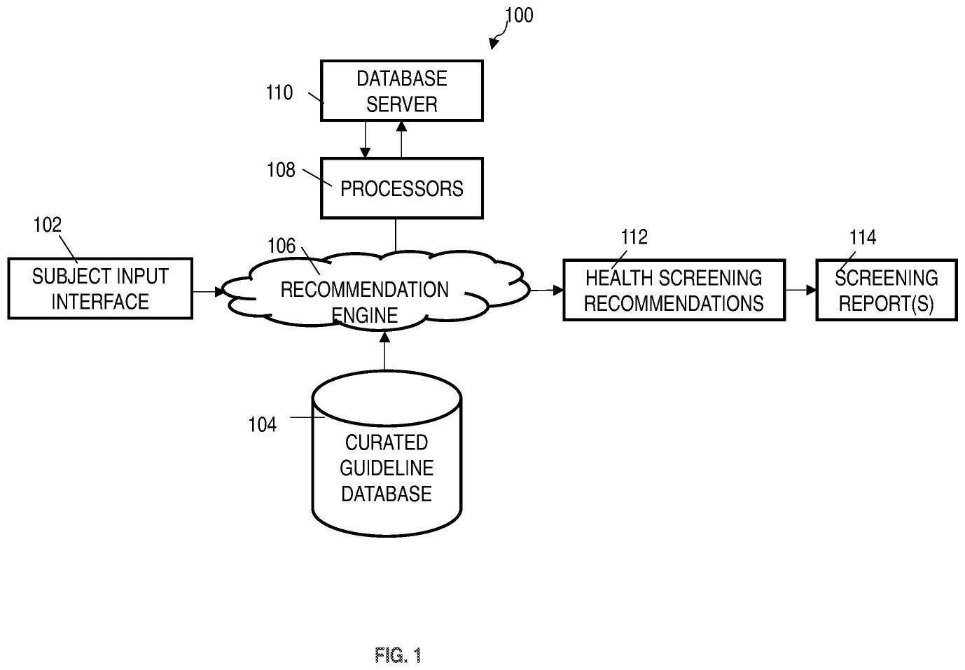

FIG. 1 illustrates a general block diagram of a system architecture of an exemplary automated personalized health screening recommendation system 100. As illustrated in FIG. 1, the system architecture comprises a recommendation engine 106, a curated guideline-based database 104, interactive subject input interface 102, one or more processors 108, a database server 110 hardware. The system 100 may be configured to produce health screening recommendations 112 and a screening report 114.

The system 100 may be based on one or more computer servers or drives operatively connected with each other, such as operated by the one or more processors 108 of the database server 110. In some embodiments, at least some components of the system 100 may be located remotely and connected to other components via an Internet link. The recommendation engine 106 may be designed as a dedicated computer processor or, alternatively, as a software-based set of commands or instructions to be implemented for another processor of the system 100.

The curated guideline database 104 may be designed as a standalone or an integrated component with an optional dedicated computer processor. In other embodiments, the curated guideline database 104 may be based on a non-transitory computer memory component as the invention is not limited in this regard. The curated guideline database 104 may be configured to incorporate one or more health screening guidelines developed by reputable organizations as discussed below in greater detail.

The interactive subject input interface 102 may be also made as a software-based component operating on any suitable electronic device, such as a cell phone, smartphone, smartwatch, PDA, tablet, laptop, personal computer, or alike. Some examples of the interaction with the subject are illustrated in greater detail below. In other embodiments, the input device may be organized as a question-and-answer session using verbal responses, such as for example, computer-generated voice prompts followed by a speech recognition component to interpret the answers from the subject. In yet further examples, a simple questionnaire may be presented to the subject as a fillable computer-based or even a paper form, with the subject's responses to be scanned in or otherwise entered into the system 100 using commonly known computer entry means. In further examples, an operator or a subject's caregiver may be asked to read the questions to the subject and record the answers in a manner suitable for subsequent computer entry, as the invention is not limited in this regard. Input from the subject or on behalf of the subject may be received in one or more interactive sessions and in multiple languages, followed by an optional translation into English.

The entire system 100 may be designed as a stand-alone computer or as a system with at least some or most of the components being cloud-based and connected online as the invention is not limited in this regard.

Soliciting personal data from the subject as input entry may include one or more personal information categories described below in greater detail. The recommendation engine 106 may be configured to receive personal subject data solicited through the interactive interface 102. The recommendation engine 106 may be then operated to find a match of the personal subject data against curated guideline-based subject database 104, such as to determine types, frequencies, and a suitable schedule for one or more health screening recommended for the subject. The system 100 may then operate one or more processors 108 and the database server 110 to process a cumulative set of health screening recommendations individualized for the subject. In one example, the recommendation engine 106 may produce a screening recommendation 112 and consequently, one or more screening reports 114. The cumulative report may be printed on paper or otherwise presented to the subject or the subject's caregiver. In further embodiments, the system 100 may be configured to present the report as a calendar containing a schedule of suitable health screening recommendations. In further embodiments, the report may include automatic integration with popular computer scheduling programs such as Google Calendar, Outlook calendar, and alike. In further yet embodiments, the system 100 may be configured to schedule suitable appointments for the subject to undergo the recommended health screenings as described below in greater detail.

The curated health screening guideline database 104 may be periodically updated when new or updated health screening recommendations and guidelines are published by reputable sources such as online or in print medical guideline publications generated by top science, medical, and government healthcare organizations. Once updated screening guidelines are entered into the curated database 104, the computerized system 100 of the invention may be operated to update previously issued reports for one or more subjects and communicate these updates thereto.

To resolve possible conflicts between health screening guidelines issued by different organizations, the system of the invention may be configured to select the most stringent and frequent set of health screening between available options. For example, if one set of guidelines recommends cancer screenings every three years and another one recommends the same screenings every two years for the same individual, the system may be configured to select a final cumulative guideline of recommending this screening to be performed every two years.

In addition to, or as an alternative to providing dedicated one or more computer servers and processors, the presently disclosed systems and methods may comprise a series of steps and operations that may be embodied in machine-executable software instructions. In other embodiments, hardware elements may be employed in place of, or in combination with, software instructions.

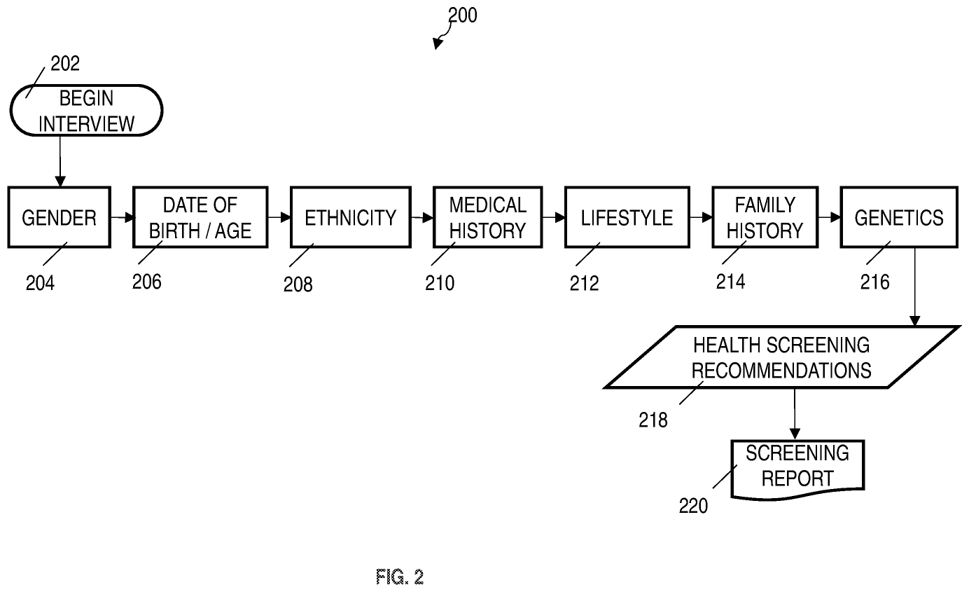

FIG. 2 illustrates the various steps of an exemplary method 200 for developing automated personalized health screening recommendations. As illustrated, method 200 begins with a subject undertaking an interview 202 through the interactive interview software interface 102 or otherwise as described above. During this interactive interview, the subject provides personal data, including personal information on one or more of the subject's personal information categories of gender 204, date of birth (age) 206, ethnicity 208, medical history 210, lifestyle 212, family history 214, and genetics 216. The resulting outcome may be a list of personal health screening recommendations 218 and a health screening report 220.

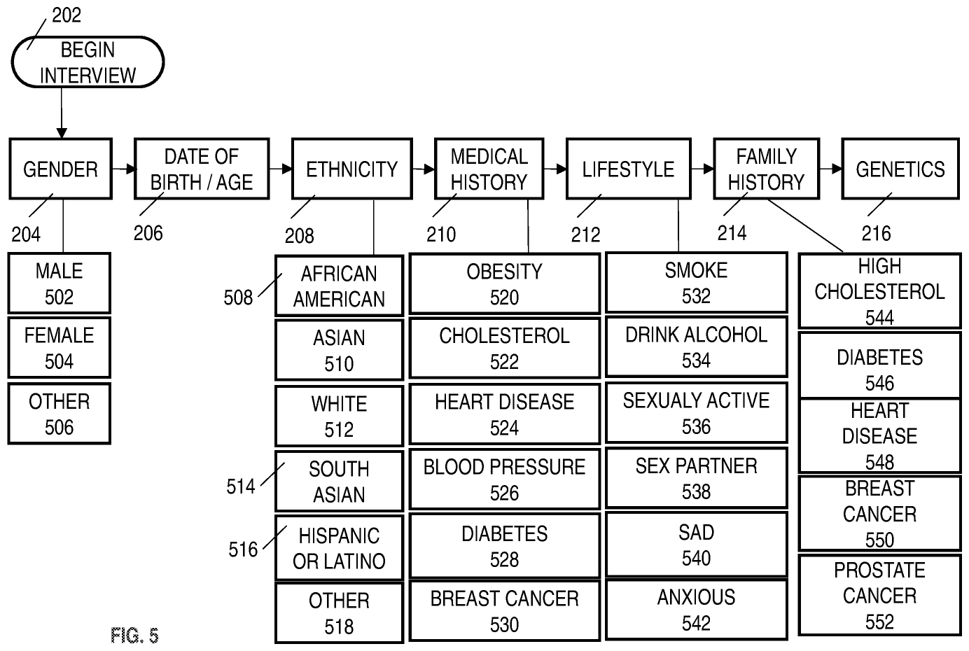

FIG. 5 illustrates how one or more personal information categories may include additional personal data and additional categories of data. For example, medical history 210 may include additional personal information categories of heart disease history 524, diabetes 528, blood pressure 526, cancer health history 530, family history 214 may include personal information of cancer 550, 552, etc. In another example, personal lifestyle history 212 may include a variety of health-related lifestyle subcategories, including sexual activity 536, 538, mental health 540, 542, a history of alcohol 534, substance and tobacco consumption 532, etc.

In at least some embodiments of the invention, a history of prior health screenings may be also solicited, for example for a predetermined past period of time such as at least one year. Once this information is received, the cumulative personalized health screening recommendation may be adjusted to avoid duplicative screenings or screenings performed shortly after another similar screening may have taken place in recent months.

In further embodiments, a history of prior health screenings and results thereof may also be solicited and used to adjust the cumulative report. If a recent screening identified a higher risk of cancer, in one example, future schedule of cancer screenings may be made more frequent for this particular subject.

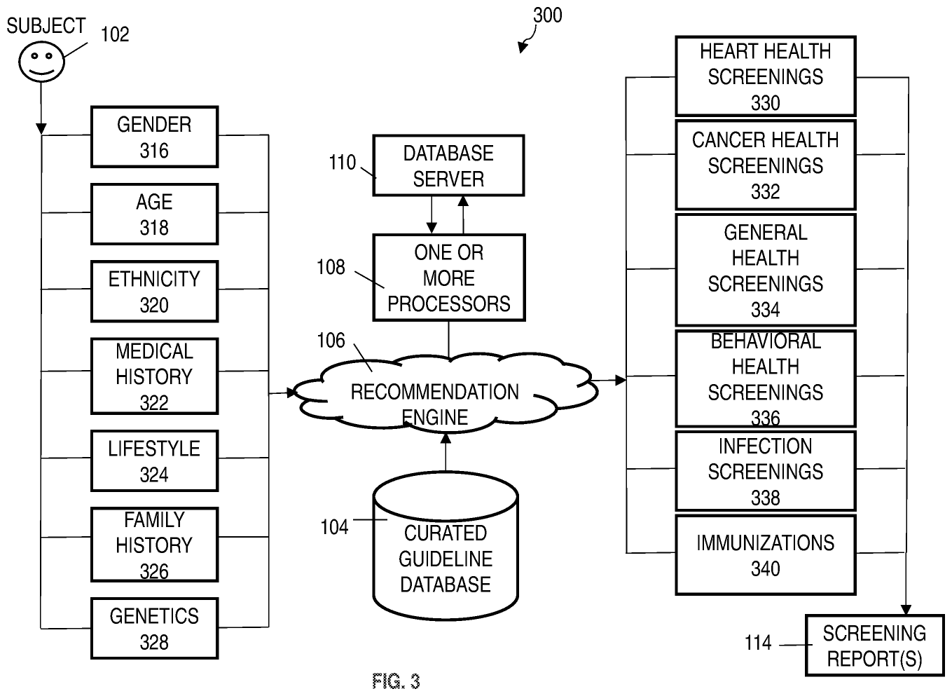

FIG. 3 illustrates a more detailed block diagram of system elements of another exemplary automated personalized health screening recommendation system 300. Subject interview 102 includes input on one or more personal information categories of gender 316, date of birth (age) 318, ethnicity 320, medical history 322, lifestyle 324, and family history 326, and genetics 328. Health screenings recommendations may be structured by one or more health category components, such as heart health screenings 328, cancer health screenings 330, general health screenings 332, behavioral health screenings 324, infection screenings 336, and immunizations 338.

The recommendation engine 106, in this case, may receive input of personal data directly solicited from the subject through interview interface 102, find a match of that data against curated guideline-based subject database 104, process recommendations on cloud-based or directly connected one or more processors 108 and a database server 110, and produce a screening report 114 with one or more screening categories 330-340.

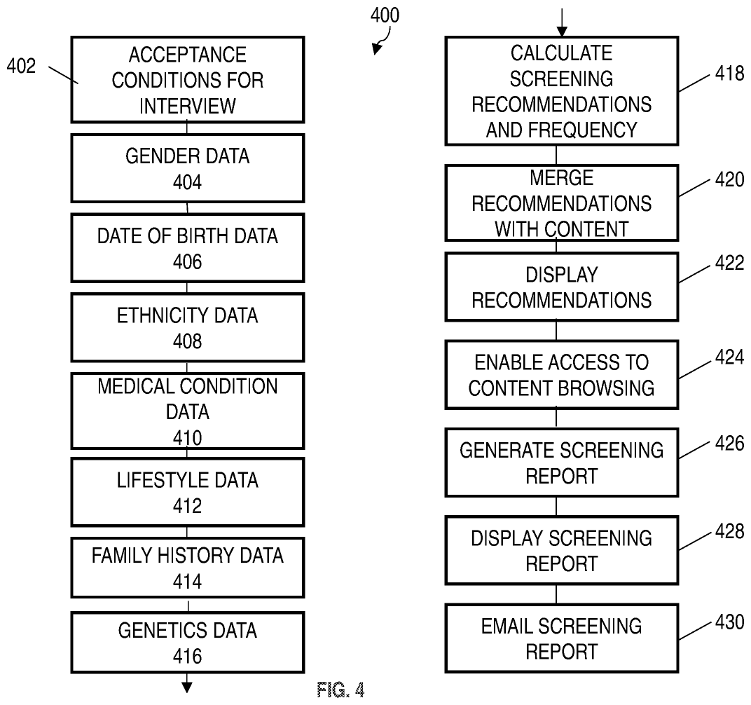

FIG. 4 illustrates a flow diagram of another method of automated personalized health screening recommendations. The system 400 may solicit and receive subject's acceptance of terms and conditions of the interview 402, followed by one or more personal information categories such as gender data 404, date of birth data 406, ethnicity data 408, medical condition data 410, lifestyle data 412, family history data 414, and genetics data 416. Each successive set of questions may be adjusted based on answers and personal information entered for prior sections of the interview. For example, if the age of the subject indicates a child, medical history and lifestyle questions may be adjusted for that of a child. Similarly, if gender is indicated to be that of a female or male, questions may be adjusted for female and male respectively. Exemplary additional details for the interview interaction and selection choices for each of these interview areas are embodied in FIG. 5.

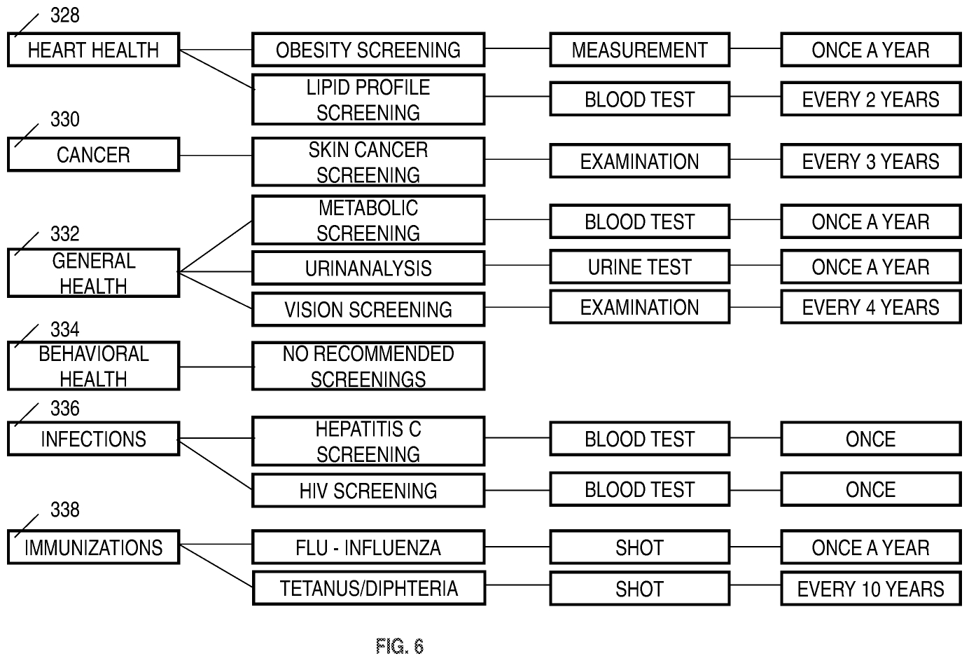

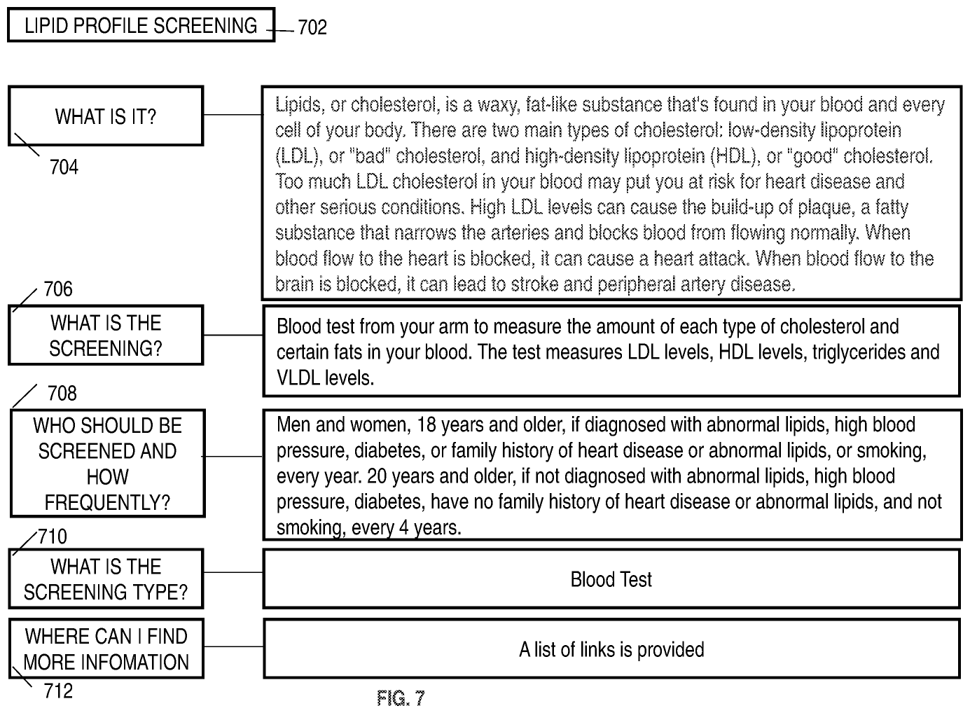

Once the last interview data is received, the recommendation engine may be operated to seek an appropriate match to the personal information of the subject using the curated guidelines database and determine the types, and the corresponding schedule for personalized screenings including screening frequency, followed by the optional merge of recommendations with screening educational content. A cumulative set of recommendations may then be presented to the subject, as illustrated in FIG. 6, including a report by health categories such as heart health, cancer, general health, behavioral health, infections, and immunizations as in one example. Educational content information may be presented as illustrated in FIG. 7, exemplified by lipid profile screening 702, including information on what the screenings is 704, what is the method for screening 706, who should be screened and how frequently 708, what is the screening type 710 and where to find additional information 712.

A more detailed logic for selecting the appropriate recommendations is discussed in Table 1 below including the use of at least one Boolean operator.

TABLE 1
An example of algorithmic steps according to the present invention

| Algorithmic Steps | Simple Screening Example |
|---|---|
| A screening guideline is identified, curated, | Obesity screening guideline: Obesity |
| classified as “strongly recommended” and | screening is strongly recommended for men |
| described in a textual format | and women, 18 years and older, every year |
| Screening type is assigned to the screening | SCREENINGTYPE = OBESITY |
| guideline | |
| Variables representing personal information | GENDER |
| are extracted from the textual description of | AGE |
| the guideline | FREQUENCY |
| Values of the variables are extracted from | GENDER = MEN OR WOMEN |
| the textual description of the guideline | AGE =< 18 |
| | FREQUENCY = EVERYYEAR |
| Values of the variables are adjusted to | GENDER = MEN or WOMEN or OTHER |
| target system specification | AGE => 18, AGE =< 80 |
| | FREQUENCY = EVERYYEAR |
| Screening type is checked if available as | SCREENINGTYPE = OBESITY COMPARE |
| type of personal medical history | TO MEDICALHISTORY = OBESITY |
| A Boolean equation is constructed to | SCREENINGTYPE = IF (MEN = 1 OR |
| represent the textual screening guideline | WOMEN = 1 OR OTHER = 1) AND (18 < |
| | AGE < 80) AND |
| | MEDICALHISTORY/OBESITY = 0); |
| | FREQUENCY = EVERYYEAR |
| After completion of the subject interview all | MEN = 1 |
| personal information variables and values | AGE = 32 |
| are parsed against the variables in the | MEDICALHISTORY/OBESITY = 0 |
| screening guideline database and extracted | |
| as variables | |
| Extracted variables are passed to the | SCREENINGTYPE/OBESITY = 1 |
| Boolean equation of the screening | |
| guideline and the equation gets solved | |
| Frequency value is merged with the | FREQUENCY = 1 |
| screening type | |
| Additional data fields are merged to the | DESCRIPTION, RATIONALLE, |
| screening type | SCREENINGTYPE, |
| | LINKSTOINFORMATION, LASTTEST, |
| | LASTTESTVALUE |
| Merged information is stored, formatted | |
| and presented to the user | |

In a more complex screening guideline, such as Lipid Profile Screening, for example, the sequential computational model may remain the same. However, the increased number of inputs and conditions may result in a more complicated Boolean function with an increased number of variables and branches. As a result, a computerized solution to recommendations becomes the only viable approach to personalized automation of screening recommendations.

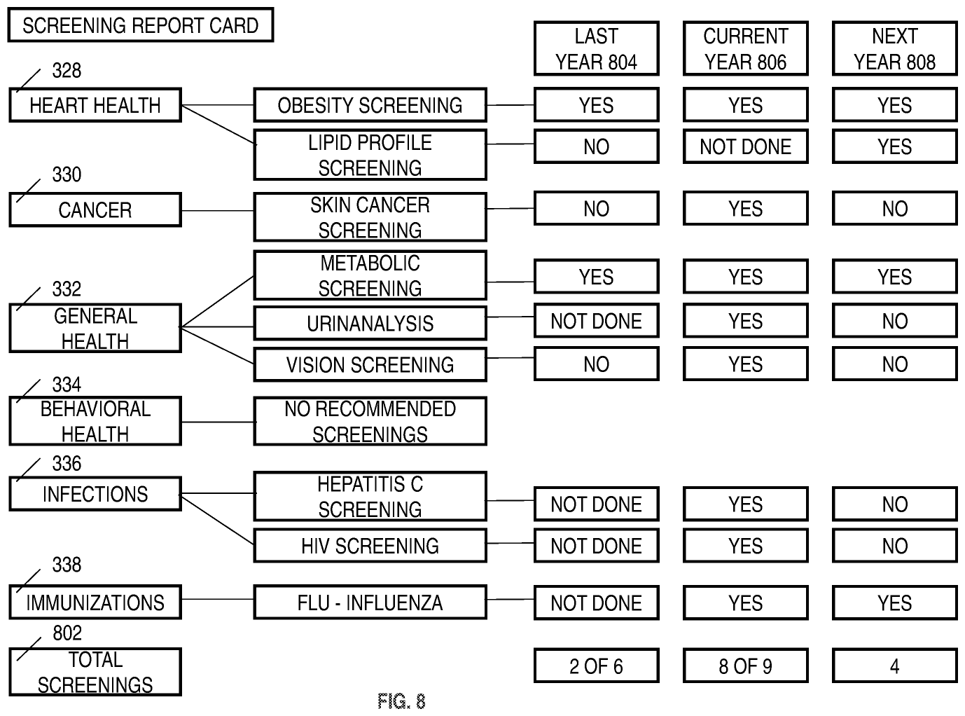

In addition to providing the subject with an individualized report on the most appropriate health screenings, the system and the method of the invention may be further configured to follow up with the subject and track the progress of the subject undertaking the recommended screenings. FIG. 8 illustrates a screening report card with screening progress tracked for last year 804, current year 806 and screenings recommended for next year 808. The total number of performed screenings may be tracked. In one embodiment, the method may include steps of providing certain incentives to the subject for performing recommended screenings or achieving a certain percentage of recommended screenings. Examples of such incentives may include free or reduced fee gym memberships, reduced fee purchases of exercise equipment and supplies, health food and supplement discounts, as well as direct cash incentives. Such incentives may be paid for by subject's employer, health insurance company, subject's family, and other beneficiaries of keeping the subject in good health.

In other embodiments, the method may include steps of soliciting further input from the subject or a healthcare provider including the types of and the results of performed recommended screenings. Accumulation of this additional information may be used to provide appropriate adjustments to the types and schedules of further screenings as the personal information for the subject changes. In addition, once anomalies are identified in the health screening results, a notification may be sent to the subject or the subject's personal physician with prompts to seek further medical attention.

In yet other embodiments, the system may be configured to automatically schedule and notify the subject of the upcoming screenings appointments. Such scheduling may be provided based on information related to the subject's geographical location, geographical location and proximity to the subject of suitable health care facilities and screening providers, medical insurance coverage information, costs of screenings, as well as subject's self-entry of user preferences, such as available hours and days of the week, preferred provider, etc.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, Aft AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, Aft BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for providing a personalized health screening recommendation for a subject, the method comprising the steps of:
a. providing a computerized recommendation engine operatively connected to a curated guideline database configured to match personal data input to a plurality of health screening guidelines,
b. soliciting personal data of the subject as input entry, the personal data comprising at least one personal information category selected from a group of personal information categories consisting of age, gender, ethnicity, personal medical history, family history, lifestyle, and genetics,
c. operating the recommendation engine to automatically process personal data of the subject solicited in step (b) against the curated guideline database to determine types and schedules of individual health screenings personally recommended for the subject, and
d. combining types and schedules of individual health screenings from step (c) to provide a cumulative personalized health screening recommendation for the subject.

2. The method as in claim 1, wherein the curated guideline database in step (a) is operated to update health screening guidelines upon availability of updates thereof from a plurality of health organizations.

3. The method as in claim 1, wherein step (b) further comprising a step of soliciting a history of prior health screenings of the subject, and wherein step (c) further comprising a step of adjusting the types and schedules of individual health screenings based on the history of prior health screenings of the subject.

4. The method as in claim 3, wherein the history of prior health screenings of the subject is solicited for at least a prior period of one year.

5. The method as in claim 1, wherein step (d) further comprising a step of structuring the cumulative personalized health screening recommendation by health categories.

6. The method as in claim 5, wherein the health categories comprise at least one health category selected from a group consisting of heart health, cancer, general health, behavioral health, infections, and immunizations.

7. The method as in claim 1, wherein the cumulative personalized health screening recommendation is a report containing a schedule and frequency of individual health screenings recommended for the subject.

8. The method as in claim 1, wherein step (d) further comprising a step of proving educational content about a nature of at least one, some, or all individual health screenings recommended for the subject.

9. The method as in claim 1, wherein the step (c) of operating the recommendation engine comprises a step of processing at least one Boolean equation by the recommendation engine for at least one individual health screening.

10. The method as in claim 1, wherein step (d) further comprising a step of providing the subject with one or more predetermined incentives to comply with the cumulative personalized health screening recommendation, wherein the one or more incentives are provided to the subject upon completion of specified health screenings.

11. The method as in claim 1 further comprising a step (e) of tracking subject compliance with recommended health screenings.

12. The method as in claim 11, wherein the step (e) of tracking further comprises a step of identifying anomalies in health screening results and prompting the subject to follow-up the identified anomalies with a medical professional.

13. The method as in claim 11 further comprising a step of adjusting the cumulative personalized health screening recommendation upon updating the personal data of the subject or upon detecting anomalies in health screening results.

14. The method as in claim 11, wherein the step (e) further comprising a step of automatically scheduling health screening appointments for the subject according to the cumulative personalized health screening recommendation of step (d).

15. The method as in claim 14, wherein the step of automatic scheduling for health screening appointments is conducted based on geographical location of the subject, geographical locations of suitable health care facilities, medical insurance information of the subject, and medical costs of performing upcoming health screenings.

16. The method as in claim 11, wherein step (e) further comprising a step of issuing an updated cumulative personalized health screening recommendation upon availability of updates for individual health screening guidelines.

17. A computer system for providing a personalized health screening recommendation for a subject, the computer system comprising:
a. at least one processor operatively connected to a computer memory, the computer memory comprising computer-readable program code embodied in the computer memory and executable by the at least one processor to perform operations of a computerized recommendation engine,
b. a curated guideline database configured to match personal data input to a plurality of health screening guidelines, the curated guideline database is operatively connected to the at least one processor,
wherein the computer-readable program code is further configured to perform the following operations:
i. record personal data of the subject as input entry via user interface of the at least one processor, the personal data comprising at least one personal information category selected from a group of personal information categories consisting of age, gender, ethnicity, personal medical history, family history, lifestyle, and genetics,
ii. operate the recommendation engine to automatically process personal data of the subject against the curated guideline database to determine types and schedules of individual health screenings personally recommended for the subject, and
iii. combine types and schedules of individual health screenings from determined in the preceding step to provide a cumulative personalized health screening recommendation for the subject.

18. A non-transitory computer-readable storage medium comprising computer-readable program code embodied in the medium that is executable by at least one processor to perform operations comprising:
a. recording personal data of the subject as input entry, the personal data comprising at least one personal information category selected from a group of personal information categories consisting of age, gender, ethnicity, personal medical history, family history, lifestyle, and genetics,
b. operating a computerized recommendation engine operatively connected to a curated guideline database to match personal data input from the preceding step to a plurality of health screening guidelines so as to determine types and schedules of individual health screenings personally recommended for the subject, and
c. combining types and schedules of individual health screenings from the preceding step to generate a cumulative personalized health screening recommendation for the subject.
